(19) United States
(12) Patent Application Publication
(10) Pub. No.: US 2025/0006317 A1
(43) Pub. Date: Jan. 2, 2025

(54) CLINICAL TRIAL OPTIMIZATION

(71) Applicant: CureMatch, Inc., San Diego, CA (US)

(72) Inventors: Alla PERLINA, San Diego, CA (US); Razelle KURZROCK, San Diego, CA (US)

(21) Appl. No.: 18/712,233

(22) PCT Filed: Nov. 23, 2022
(86) PCT No.: PCT/US2022/080457

§ 371 Date: May 21, 2024

Related U.S. Application Data
(60) Provisional application No. 63/282,685, filed on Nov. 23, 2021.

(51) Int. Cl. G16H 10/20; G16H 10/40; G16H 10/60; G16H 15/00; G16H 50/70

(52) U.S. Cl. CPC G16H 10/20; G16H 10/40; G16H 10/60; G16H 15/00; G16H 50/70

(57) ABSTRACT

The present disclosure generally relates to methods and systems for improving personalized medicine. In one aspect, a method for assessing eligibility of a patient to a clinical trial is provided based on evaluation of molecular profiles of patients and matching them with the predicted response to a therapy tested in the clinical trial. Embodiments include determining impact of a therapy tested in the clinical trial on the molecular profile of the patient based on the number and types of biomarkers and their mutual interaction between each other and drugs of the therapy.

### CROSS-REFERENCE TO RELATED APPLICATIONS

This application claims priority from U.S. Provisional Application Ser. No. 63/282,685 filed on Nov. 23, 2021, which is incorporated herein by reference in its entirety.

### FIELD

The present disclosure generally relates to methods and systems for improving personalized medicine, and more specifically to assessing eligibility of patients to a clinical trial based on molecular profiles of patients.

### BACKGROUND

Personalized medicine involves the use of biomarkers to guide treatment regimens adapted to each patient. Traditional clinical trial enrollment comprises qualifying (or inclusion) criteria defining patient characteristics of who may be eligible to participate in a specified clinical trial, and disqualifying criteria defining patient characteristics of who are not eligible for participation in the clinical trial. For example, inclusion criteria may comprise the disease or condition that the clinical trial is addressing, previous treatments and medical history of a patient, and so on. Disqualifying criteria may comprise, for example, previous treatments that disqualify a patient from participating in the clinical trial, a stage of a disease beyond which a patient would be ineligible for inclusion into the trial, and so on. However, typical inclusion and disqualifying criteria do not take into account full molecular profile of a patient, which results in potential inclusion of broad categories of patients, from which only a fraction will truly benefit from such inclusion. There remains a need for techniques to inform healthcare providers or pharmaceutical companies of the most risky subset of profiles to potentially exclude from the trial and to select an optimal cohort of patients who will have increased chances of responding to the therapy and/or decreased chances of experiencing toxicity in the clinical trial. The present invention addresses this and other needs.

### SUMMARY

Typically, to determine if a patient is eligible for a participation in a clinical trial, the patient's doctor and/or other clinical staff will review the patient's medical records and the qualifying and disqualifying criteria set for the clinical trial. When disease or condition addressed by the clinical trial is complex and/or multifactorial, multiple clinically relevant biomarkers may potentially be present in patients, but only a fraction of these biomarkers may be targeted by the therapy tested in the clinical trial. Therefore, depending on abundance of non-targeted biomarkers in different patients, these patients will be more or less benefited from the current setup of the trial. Inclusion of a significant percentage of patients who will have little or no benefit from the clinical trial could obscure trial results and jeopardize the whole drug development program. In some embodiments, the present disclosure provides a method for selecting an optimal cohort of patients whose molecular profiles match to a certain extent to biomarkers targeted by predicted response to the therapy tested in the clinical trial.

In one embodiment, provided herein is a method for assessing eligibility of a patient to a clinical trial of a therapy comprising one or more drugs, the method comprising:
(a) providing at least one drug target for the one or more drugs, and providing a molecular profile for the patient, wherein the molecular profile is generated from analysis of a biological sample obtained from the patient and contains one or more clinically relevant biomarkers;
(b) generating a matching score for the patient based on the molecular profile and the patient's predicted response to the therapy, and setting a threshold score, wherein the generating step comprises determining impact of the therapy on the molecular profile of the patient based on the number and types of biomarkers and interactions between biomarkers and the one or more drugs of the therapy;
(c) providing a recommendation for clinical trial eligibility of the patient when the matching score for the patient passes, meets or exceeds requirements of the threshold score.

In another embodiment, provided herein is a system for assessing eligibility of a patient to a clinical trial of a drug, the system comprising:
a server;
a computing device in communication with the server over a network, the computing device including a processor and a memory, the memory bearing computer executable code configured to perform the steps of:
(a) providing at least one drug target for the one or more drugs, and providing a molecular profile for the patient, wherein the molecular profile is generated from analysis of a biological sample obtained from the patient and contains one or more clinically relevant biomarkers;
(b) generating a matching score for the patient based on the molecular profile and the patient's predicted response to the therapy, and setting a threshold score, wherein the generating step comprises determining impact of the therapy on the molecular profile of the patient based on the number and types of biomarkers and interactions between biomarkers and the one or more drugs of the therapy;
(c) providing a recommendation for clinical trial eligibility of the patient when the matching score for the patient passes, meets or exceeds requirements of the threshold score.

These and other features, aspects and advantages of the present teachings will become better understood with reference to the following description, examples, and appended claims.

### BRIEF DESCRIPTION OF THE DRAWINGS

The foregoing and other objects, features and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein

FIG. 1 illustrates an exemplary therapy matching system. The therapy matching and scoring system consists of curated knowledge base component that allows the algorithms to programmatically match an incoming cancer patient's profile against the relevant structured knowledge for each unique case. Inputs include but are not limited to cancer NGS testing report, diagnosis, trial drug(s) and their target(s). The scoring AI engine uses the relevant content, reasoning algorithms, and rules to produce matching scores for each patient in each arm of a trial. Scores can be used to establish thresholds for the population.

FIG. 2 illustrates an exemplary clinical trial therapy matching process. The process of matching patients necessitates that cancer samples are obtained from the patients and sent for molecular lab testing capable of detecting cancer-specific DNA biomarkers and optionally other data types. The lab reports are used an inputs for therapy scoring inside the computer system, which subsequently generates reports for each patient or a patient population.

FIG. 3 illustrates an exemplary system output. The report generated by the system contains (but not limited to) de-identified patients' results with scores for each arm of the trial. Horizontal arrow in Arm2 result of patient ID 14 points to an example of a score that may result in deeming the patient as ineligible, because the set threshold requirement was not met, i.e., in this example, the score of 0 is below 5^{th} percentile threshold.

FIG. 4 illustrates an exemplary eligibility determination for a clinical trial that investigates a drug A.

When a monotherapy (Drug A) is evaluated in a clinical trial targeting a cancer Marker A as a direct and exclusive target, subjects like Patient 1 who have Marker A alone or along with 1 other mutation (Marker B) might have a high score of 50% or above (depending on the nature of the alteration in Marker B), whereas in the Patient 2 scenario, where additional known oncogenic/pathogenic biomarkers are present (C, D, E) would result in much lower scores, because Drug A would be at best addressing only 20% of the cancer driving profile (1 out of 5 biomarkers). In this simplified representation of the matching and scoring concept, no additional AI reasoning takes place to account for either specific exclusionary logic based on the nature of other possibly relevant drug-target relationships or marker types (C-E) or direct or indirect mechanistic targeting pathways or IC50 values for the drug-target interaction(s) (maximum sensitivity is assumed for simplicity).

FIG. 5 illustrates an exemplary system computational architecture. Specifically, the figure shows a functional block diagram of a system architecture including the physical location of various components. The system may include an interface side and a computational side. In an aspect, there are at least two machines, with at least one in each category. The interface side may include a software-based user interface connected to a storage database. A user may interact with the system by inputting patient data into the user interface, requesting jobs (e.g., making requests for computational jobs), and searching for stored data (e.g., searching and/or viewing data from the stored database in a human-readable format). Job requests may contain user-defined parameters and inputted data. Job requests may be sent from the interface to the decision support system on the computational side. The computational side may include a score generation system, a knowledge database, and a working database meant to store the flow of data generated during the computation processes. In other words, the computational side may utilize a knowledge database including curated biological data, as well as a working database meant to store data generated during the computational processes. The decision support system may read data from the knowledge database, and read and write data generated as a byproduct of the combinatorial computations into the working database. The decision support system final output (e.g., eligibility recommendation) may be sent back to the interface server and stored into the storage database.

FIG. 6 illustrates an exemplary user's data input. The figure shows the origin and the type of patient data that may be entered into Module 1 of a system. By way of example, a patient may visit a healthcare provider that records medical history and collects a sample suitable for molecular analysis. Molecules of interest (e.g., DNA, RNA, proteins, or the like) may be extracted from the sample and sent to a specialized laboratory to perform molecular profiling. Molecular results, clinical data, and user-defined parameters may be inputted in the system using a user-based interface.

Thus, in a first step, data may first be obtained. Using the example above, a patient may visit a physician, who obtains and records informative medical and demographics data (e.g. age, gender, histological diagnosis, previous treatment(s), current treatment, medical history and comorbidities, familial history and inherited syndromes, and the like) and performs a biopsy (tumor, blood, tissue of interest, body fluid, or the like) in order to define the status of biomarkers specific to the patient's disease. The status of biomarkers can be obtained from a validated third party company or an in-house laboratory, and can be assessed using methods of genomic profiling (e.g., DNA sequencing, comparative genomic hybridization, in situ hybridization, and the like), epigenomics profiling (e.g., histone modification assay, chromatin immunoprecipitation, restriction landmark analysis, bisulfite sequencing, and the like), transcriptomic profiling (e.g., microarray, RNA sequencing, real-time polymerase amplification, and the like), proteomic profiling (e.g., immunoassay, mass spectrometry, and the like), metabolomics profiling, or biochemistry profiling. Next, data may be inputted. The physician (or designate) may log in to the system's web interface and enter information relevant to the system's use such as patient identification, patient diagnosis, patient treatment, and molecular description.

FIG. 7 illustrates an exemplary computer system.

FIG. 8 illustrates an exemplary cloud computing system.

### DETAILED DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to comprise items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” “substantially,” or the like, when accompanying a numerical value, are to be construed as indicating a deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the embodiments.

In the following description, it is understood that terms such as “first,” “second,” “top,” “bottom,” “up,” “down,” and the like, are words of convenience and are not to be construed as limiting terms unless specifically stated to the contrary.

As used herein, the term “biomarker” refers to a biological molecule found in blood, other body fluids, or tissues of a patient that is a sign of an abnormal process, or of a condition or disease. As used herein, the term “clinically relevant biomarker” refers to a biomarker that is pathogenic and/or actionable. Pathogenicity is determined by a clinical lab that performs molecular testing of a patient's sample and issues a report indicating which biomarkers identified in the patient's sample are pathogenic. Preferred way of performing molecular testing is by using next-generation sequencing (NGS). “Actionable” biomarker refers to a biomarker that is known to either be a direct or indirect drug target, or a known marker of sensitivity to a specific drug or drug combination.

The biomarkers used to guide personalized medicine may be of several different types. They can be derived from genomic (e.g., DNA alterations), epigenomic (e.g., DNA methylation alterations), transcriptomic (e.g., RNA alterations including but not limited to coding RNAs and non-coding RNAs), proteomic (e.g., protein alterations), metabolomic (e.g., metabolite level alterations), or biochemistry (e.g., chemicals level alterations). These biomarkers can be detected in normal/healthy tissues, pathologic tissues (such as benign or malignant tumors), or pathogen-related entities (e.g., bacteria, virus, and the like). They can also be inherited (germline molecular patterns) or arise de novo in an individual (e.g., after-birth, somatic molecular patterns) which could occur after a particular environmental exposure (e.g., tobacco smoke, pollutants, and so on).

As used herein, the term “molecular profile” for a patient refers to a list of clinically relevant biomarkers identified in one or more patient's samples.

As used herein, the term “clinical trial” refers to a clinical research study performed in people that is aimed at evaluating a safety and/or efficacy of a therapy (i.e., experimental therapy) applied against a specific disease or condition. A therapy may be a single drug, or a combination of drugs (combination therapy). A drug may be a small molecule drug, or a biological drug, such as a protein drug, or a nucleic acid drug. In some embodiments, a drug may be a vaccine. In some embodiments, drugs of different types (e.g., one or more small molecule drugs and one or more biological drugs) may be combined together in one therapy (combination therapy). Disease or condition being evaluated in a clinical trial may be in any medical field, including but not limited to oncology, immunology A patient may suffer from a proliferative disease (e.g., cancer of any type, psoriasis, benign tumors, and the like), a degenerative disease (e.g., Alzheimer's disease, Parkinson's disease, and the like), a metabolic disease (e.g., diabetes, heart diseases, neurological disorders, and the like), or an infectious disease (e.g., AIDS, viral hepatitis, bacterial meningitis, and the like), neurology, infectious disease, and psychiatry.

As used herein, the term “drug target” refers to a biological macromolecule or a biomolecular structure in the patient's body that binds to a specific drug, which produces a therapeutic effect. Often, a drug target is intrinsically associated with a disease process addressed by the drug. Non-limiting examples of drug targets include proteins, genes, pathological assemblies (such as protein aggregates), lipid oxidation products, and others.

As used herein, the terms “matching score” or “score” are used interchangeably herein and refer to an indicator of the degree to which a given therapy matches a patient's molecular profile. This includes all clinically relevant cancer biomarkers regardless of their therapeutic actionability. The more markers addressed by a given therapy (therapeutic option), the higher the matching score; complete targeting of all markers would mean ˜100% matching score; no molecular basis for matching the given therapy would mean 0% matching score. In some embodiments, the matching score is a continuous numerical value between 0 and 100 rounded to natural numbers. Various algorithms can be utilized to calculate the matching score.

The present disclosure provides a system that allows better patient stratification and clinical trial design based on the degree of molecular matching of combination- or monotherapies in consideration to each patient's molecular profile providing a unique method for determining eligibility of subjects for clinical trial enrollment. In addition, the system can also analyze previously treated or untreated patients to identify those who would benefit from the clinical trial.

In some embodiments, systems and methods for optimizing a clinical trial design and/or outcome are provided herein. The system can include a server; a computing device in communication with the server over a network, the computing device including a processor and a memory, the memory bearing computer executable code configured to perform the steps of:
identifying a drug target and biomarker of interest in a clinical trial;
obtaining individual patient data from a population of patients, wherein the patient data includes at least one biomarker specific to each individual patient;
employing a scoring engine to score each patient based on the patient's predicted response to the drug;
identifying a group of patients within the population having matching scores above a predetermined threshold to study in the clinical trial;
whereby the clinical trial design and/or outcome are optimized. The method can include identifying a drug target and biomarker of interest in a clinical trial; obtaining individual patient data from a population of patients, wherein the patient data includes at least one biomarker specific to each individual patient; employing a scoring engine to score each patient based on the patient's predicted response to the drug; and identifying a group of patients within the population having matching scores above a predetermined threshold to participate in the clinical trial; whereby the clinical trial design and/or outcome are optimized.

In some embodiments, a method for assessing eligibility of a patient to a clinical trial of a therapy comprising one or more drugs is provided, the method comprising:
(a) providing at least one drug target for the one or more drugs, and providing a molecular profile for the patient, wherein the molecular profile is generated from analysis of a biological sample obtained from the patient and contains one or more clinically relevant biomarkers;
(b) generating a matching score for the patient based on the molecular profile and the patient's predicted response to the therapy, and setting a threshold score, wherein the generating step comprises determining impact of the therapy on the molecular profile of the patient based on the number and types of biomarkers and interactions between biomarkers and the one or more drugs of the therapy;
(c) providing a recommendation for clinical trial eligibility of the patient when the matching score for the patient passes, meets or exceeds requirements of the threshold score.

In some embodiments, the threshold score has a numerical value, and a recommendation for clinical trial eligibility of the patient is provided when the matching score for the patient meets or exceeds the threshold score.

In some embodiments, the threshold score has predetermined requirements, such as requirements that are set before start of the clinical trial.

In some embodiments, determination of patient's eligibility based on the threshold score depends on the design of the trial arms and patient stratification needs of each clinical trial. In some embodiments, threshold requirements may be established in order to identify the most at-risk patients that may need to be excluded as those patients whose scores, for example, fall below the 5^{th} percentile while patients scoring at 5^{th} percentile or above would be considered as having met (or passed) the threshold requirements.

In some embodiments, different clinical trial arms necessitate different threshold requirements, where thresholds may be set to define a range of high- or low-scoring patients. In one particular embodiment, in a trial of Drug A, there is a Control arm with drug C, for which only the top 5^{th} percentile group is of interest. In such cases, subjects that pass threshold requirements may be patients that have a score percentile of more than 95. In another particular embodiment, a trial of Drug A in arm 1 is also testing a combination therapy arm 2 with Drugs A+B included. Thresholds are set at 5^{th} and at 20^{th} percentile to define the low scoring ranges and further categorize low matching patients into “low” or “very low” if needed.

In some embodiments, threshold requirements may be the same or different for each arm. In some embodiments, a strict low-scoring population at risk needs to be identified with one threshold value while another threshold value may be needed to represent a range of borderline low-scoring patients. Similarly, a range of high score values may be needed to determine the sub-population of predicted good favorable responses, as well as a group of potentially exceptional responders. In such example, an exemplary threshold range could be 90^{th}-95^{th} percentile score values, and those patients that have such scores would fall into one or the other high-scoring category as per threshold requirements.

In some embodiments, the impact of the therapy on the molecular profile of the patient is determined by determining correspondence between the at least one drug target affected by the applied therapy and the one or more clinically relevant biomarkers present in the patient. In some embodiments, the impact of the therapy is determined for each clinically relevant biomarker present in the patient. In some embodiments, when the one or more drugs of the therapy are predicted to successfully target all the clinically relevant biomarkers present in the patient, the matching score of the therapy for the patient is maximized. In other embodiments, when the one or more drugs of the therapy are predicted to successfully target only a minor fraction (e.g., only about 5%, about 10%, about 15%, about 20%, about 25%, about 30%, about 40% or about 50%) of the clinically relevant biomarkers present in the patient, the matching score of the therapy for the patient is reduced based on at least the percentage of the clinically relevant biomarkers present in the patient and predicted to be targeted by the one or more drugs of the therapy.

In some embodiments, generation of a matching score for the patient is based on the molecular profile and the patient's predicted response to the therapy. In some embodiments, the matching score is generated based at least on the percentage of the clinically relevant biomarkers present in the patient and predicted to be targeted by the one or more drugs of the therapy.

In some embodiments, determining impact of the therapy on the molecular profile of the patient comprises determining correspondence between the at least one drug target affected by the applied therapy and the one or more clinically relevant biomarkers present in the patient.

In some embodiments, drugs of the therapy target all clinically relevant biomarkers provided in the molecular profile of the patient. In some embodiments, drugs of the therapy target each of the clinically relevant biomarkers provided in the molecular profile of the patient with at least some efficiency. In other embodiments, drugs of the therapy target only some clinically relevant biomarkers provided in the molecular profile of the patient with at least some efficiency, but do not target the other clinically relevant biomarkers of the patient's molecular profile.

In some embodiments, determining impact of the therapy on the molecular profile of the patient comprises determining how well the one or more drugs of the therapy can target the provided clinically relevant biomarkers of the patient. In some embodiments, one drug may target a biomarker differently or with different efficiency compared to another biomarker. In some embodiments, targeting efficiency may be calculated from provided or calculated half maximal inhibitory concentration (IC50) of the drug from the therapy towards a clinically relevant biomarker.

In some embodiments, the molecular profile generated for the patient is agnostic to a) NGS lab and technology type; b) sample type (can be liquid or tissue biopsy); c) panel size;
and d) omics data type (genomic/transcriptomic/proteomic).

In some embodiments, during generation of the molecular profile, somatic DNA results with interpreted clinical significance/relevance are generated. In some embodiments, multiple types of tumor profiling results (DNA/RNA sequencing, protein expression, etc.) are analyzed integratively.

In some embodiments, the exemplary data elements of the molecular profile of the patient include but are not limited to the following:
a. Date of Birth (or rounded age, in years, is required);
b. Sex at birth;
c. Diagnosis;
d. Laboratory Name (lab reports with, at least, somatic DNA data required);
e. List of biomarkers/variants with their aberration types and clinical significance determination is required, such as Pathogenic, Non-pathogenic, VUS and other;
f. Optionally, other biomarkers (from NGS lab or pathology report, can be DNA, RNA or protein data).

In some embodiments, examples of therapy types that can be evaluated by the disclosed methods include: chemotherapy, immunotherapy, hormone targeting therapy or various molecular targeted therapies with small molecules and/or monoclonal antibodies.

In some embodiments, the patient is a cancer patient, and the one or more clinically relevant biomarkers are distinct for the patient's cancer.

In some embodiments, the one or more clinically relevant biomarkers is/are targeted by the one or more drugs of the therapy. In these embodiments, the one or more clinically relevant biomarkers comprise the at least one drug target.

In some embodiments, the therapy comprises two or more drugs, three or more drugs, 4 or more drugs, or 5 or more drugs. In other embodiments, the therapy consists of a single drug (monotherapy).

In some embodiments, generating the matching score for the patient based on the molecular profile and the patient's predicted response to the therapy comprises leveraging existing clinical data to uncover relationships for drugs and/or biomarkers of interest with other molecular factors using machine learning (ML) or other analytical techniques to predict relevant features that need to be considered by the system for matching and scoring results predicted to better relate to clinical outcomes. Certain elements of the system for matching and scoring results are disclosed in U.S. Pat. No. 11,434,534 B2, incorporated by reference herein.

In some embodiments, the disclosed method further comprises providing a list of features most likely related to success or failure of the clinical trial. In some embodiments, the disclosed method further comprises providing clinical trial design alternative, such as different therapy arms or specific molecular inclusion or exclusion criteria for the subsequent trial phase or for a new trial planning.

In preferred embodiments, each drug of the therapy has one or more drug targets. In some embodiments, a drug of the therapy has two or more, three or more, 4 or more, or 5 or more drug targets.

In some embodiments, the therapy comprises two or more drugs, and drug targets for each drug of the therapy are different from each other. In other embodiments, the therapy comprises two or more drugs, and drug targets for each drug of the therapy are at least partially overlap.

In some embodiments, eligibilities of a plurality of patients (e.g., group of patients) are assessed, and matching scores are generated for each patient of the plurality of patients, forming collectively a range of matching scores for the plurality of patients.

In some embodiments, the threshold score is set after a matching score for each patient of the plurality of patients is generated.

In some embodiments, the threshold score is set at a specific percentile of a range of matching scores generated for each patient of the plurality of patients.

In some specific embodiments, the threshold score is set at the fifth percentile of matching scores generated for each patient of the plurality of patients. In other specific embodiments, the threshold score is set at the tenth or other percentile of matching scores generated for each patient of the plurality of patients.

In some embodiments, the threshold score is set before a matching score for each patient of the plurality of patients is generated.

In some embodiments, the threshold score is set based on previously analyzed clinical trial data sets.

In some embodiments, setting the score threshold can be based on a newly obtained patient data from the clinical trial, published or proprietary real-world evidence data from past clinical studies, or other previously analyzed clinical molecular data. For example, there may very few gastric cancer samples in a given clinical trial cohort, and the pharmaceutical company or other company interested in determining relevance and thresholds of therapy of interest across a bigger population, may opt to rely on previously processed gastric cancer data or combine multiple datasets into one.

In some embodiments, more than one threshold for scores is set. Each therapy of interest envisioned for the trial can be considered as a separate arm with 1 or more drugs. Each clinical therapy arm of a trial can have 1 or more thresholds or threshold ranges, depending on the nature and the size of the data. For example, for some arms of a trial only 5^{th} percentile score is of interest for thresholding whereas in another arm there may be 5^{th} and 95^{th} percentile scores to establish the exceptionally poor and or exceptionally great matching population subsets. A threshold range may be required for some populations. For example, to identify the strictly low-scoring subpopulation from more of a borderline-low matching scores, the analysis can identify the thresholds and the related patients that fall into 5^{th}-20^{th} score percentiles.

In some embodiments, a method for assessing eligibility of a group of patients to a clinical trial of a therapy comprising one or more drugs is provided, the method comprising:
(a) providing at least one drug target for the one or more drugs, and for each patient from the group of patients providing a molecular profile, wherein the molecular profile is generated from analysis of a biological sample obtained from the patient and contains one or more clinically relevant biomarkers (i.e., markers of known cancer causing or disease promoting nature, or markers of sensitivity or resistance to any drugs that are used for disease targeting);
(b) generating a matching score for each patient from the group of patients based on the molecular profile and the patient's predicted response to the therapy, and setting a threshold score, wherein the generating step comprises determining impact of the therapy on the molecular profile of the patient based on the number and types of biomarkers and their mutual interaction between each other and drugs of the therapy;
(c) providing a recommendation for clinical trial eligibility for patients having the matching score above the threshold score.

In some embodiments, the threshold score is set at the 5^{th}, 10^{th}, 15^{th}, 20^{th}, 25^{th}, 30^{th}, 40^{th}, or 50^{th} percentile of matching scores generated for each patient of the group of patients.

In some embodiments, the providing at least one drug target for the drug comprises providing an IC₅₀ value of the drug for the at least one drug target.

In some embodiments, the biological sample comprises a blood, a plasma or a serum sample.

In some embodiments, the clinical trial is a prospective clinical trial.

In some embodiments, the clinical trial is a retrospective analysis of past trials' data or historical data, or otherwise available clinical Real World Data analysis.

In some embodiments, the analysis of the biological sample comprises performing a next-generation sequencing of nucleic acid molecules extracted from the biological sample.

In some embodiments, the analysis of the biological sample further comprises extracting a cell-free DNA from the biological sample. In some embodiments, the analysis of the biological sample further comprises evaluating the following molecular additional data types: RNA, protein, metabolites/metabolome, immunome, microbiome, epigenetic, pharmacogenetic/pharmacogenomic and/or other data types.

In some embodiments, generating the matching score further comprising determining impact of the drug on each of the one or more clinically relevant biomarkers.

In some embodiments, interactions between biomarkers and the one or more drugs of the therapy are determined from expert-curated knowledge base and may be updated as new literature emerges or new drugs get approved.

In some embodiments, the knowledge base contains structured expert-curated peer-reviewed evidence across multiple clinical science domains pertinent to precision oncology, such as:
i. clinical genomics (to assure correct matching of clinically relevant cancer markers);
ii. pharmacology (to equip the reasoning and scoring engine with information on which markers can be targeted by which drugs and how strong and specific each drug-target interaction is, based on IC50 values and other available content);
iii. drug mechanisms (to equip the reasoning and scoring engine with information on direct and indirect targeting of cancer biomarkers by each drug);
iv. information from drug labels (may include not only the information from points (i), (ii), and (iii), but also specific disease and patient's age and gender context, and other content;
v. clinical trials;
vi. resistance and toxicity, pertaining to each patient's molecular biomarkers, to allow the system to exclude drugs or combinations to which there can be harmful effects, based on peer-reviewed literature about cancer therapies and biomarkers, published clinical outcomes, FDA labels and NCCN guidelines.

In some embodiments, interactions can be
between biomarker(s) and drug(s) of the therapy,
between 2 or more biomarkers, and/or
between 2 or more drugs of the therapy.

In some embodiments, interactions between biomarkers and the one or more drugs of the therapy can be of positive or negative nature, i.e., biomarker of sensitivity or resistance to some drug(s); biomarker that if occurs together with other specific biomarkers would have different impact on inclusion/exclusion of certain drugs into the therapeutic options and the respective scores. In some embodiments, drug-drug interaction can be synergistic or required for some molecular profiles. In some embodiments, drug-drug interaction can be toxic or lead to hyperprogression of disease.

In some embodiments, there is a direct interaction between a biomarker and a drug of the therapy, which implies that the drug is evidenced to molecularly target the specific biomarker.

In some embodiments, there is an indirect interaction between a biomarker and a drug of the therapy, which implies that the biomarker is not a direct target of the given drug, but the drug is evidenced to pharmacologically or clinically address the marker via documented biological pathway or process interactions. For example, KRAS G12V mutation is known to be often addressed by inhibiting the associated downstream biological pathway called MEK or MAPK pathway with drugs like trametinib, binimetinib, or other MEK1/2 inhibitors.

In some embodiments, there is an indirect interaction between a biomarker and a drug of the therapy, which implies that the given biomarker is demonstrated as a biomarker of sensitivity to a given drug even if the precise molecular mechanism of action of the drug is aimed at a distinct target and not intended to interfere with the biomarker in any way. For example, high tumor mutation burden (TMB) is a biomarker of sensitivity to checkpoint inhibitors, which are drugs like pembrolizumab (Keytruda) that target the PD-L1/PD-1 pathway and are not intended to target the tumor mutation burden itself in any way.

In some embodiments, biomarkers comprise genomic, proteomic, transcriptomics, epigenetic, metabolomic, and various immune system markers, such as PD-L1 or neoantigen markers, where functional effects may include but are not limited to immunogenicity, sensitivity or resistance to drugs that may include but are not limited to targeted molecular therapies and biosimilars, chemotherapies, hormone-targeting therapies, and immunotherapies, including check-point inhibitors, CAR-T therapies, and personalized vaccines analyzed as potentially matching monotherapy or combination therapy options.

In some embodiments, biomarkers are obtained via at least one of: a biopsy from tissue or blood; an external resource; transcriptomic profiling, wherein the transcriptomic profiling includes one or more of a microarray, RNA sequencing, real-time polymerase amplification; genomic profiling, wherein the genomic profiling includes one or more of hereditary or cancer DNA results from laboratory testing such as Next Generation Sequencing (NGS), PCR, histone modification assay, chromatin immunoprecipitation, restriction landmark analysis, bisulfate sequencing, cancer neoantigen analysis results, T-Cell and other immune cell sequence analyses, clonality, or heterogeneity; proteomic profiling, wherein proteomic profiling includes one or more of an immunoassay and mass spectrometry, immune markers, which may include cancer neoantigens; and one or more of metabolomics profiling and biochemistry profiling.

In some embodiments, a system for assessing eligibility of a patient to a clinical trial of a drug is provided, the system comprising:
a server;
a computing device in communication with the server over a network, the computing device including a processor and a memory, the memory bearing computer executable code configured to perform the steps of:
(a) providing at least one drug target for the one or more drugs, and providing a molecular profile for the patient, wherein the molecular profile is generated from analysis of a biological sample obtained from the patient and contains one or more clinically relevant biomarkers;
(b) generating a matching score for the patient based on the molecular profile and the patient's predicted response to the therapy, and setting a threshold score, wherein the generating step comprises determining impact of the therapy on the molecular profile of the patient based on the number and types of biomarkers and interactions between biomarkers and the one or more drugs of the therapy;
(c) providing a recommendation for clinical trial eligibility of the patient when the matching score for the patient passes predetermined requirements of the threshold score.

In some embodiments, the disclosed system is configured to provide a list of features most likely related to success or failure of the clinical trial. In some embodiments, the disclosed system is configured to provide a clinical trial design alternative, such as different therapy arms or specific molecular inclusion or exclusion criteria for the subsequent trial phase or for a new trial planning.

In some embodiments, a scoring engine or an algorithm is employed to generate a matching score for each patient based on the patient's predicted response to the therapy.

In some embodiments, the algorithm used to generate a matching score incorporates the knowledge in the form of numerous explicit rules, which are otherwise not generalizable, but are required to assist in “doing no harm” and addressing unique molecular profiles of the patients. In some embodiments, the algorithm runs any relevant combination of biomarkers and targets, including never-before-seen cancer drug targeting profiles, against the knowledge base. After that the algorithm calculates therapy matching options, then filter, and score them for a generated report to a user, such as service provider, a pharmaceutical company's representative, or a doctor. The user then considers the scores for the clinical cohort and can propose one or more score threshold(s) to decide for which patients the trial therapy is most clinically appropriate and for which patients certain molecular enrollment criteria need to be reconsidered before enrolling into the trial.

In some embodiments, generating a matching score for the patient based on the molecular profile and the patient's predicted response to the therapy utilizes an algorithm similar to one used in a Global Positioning System (GPS) Navigation device, which is a type of Artificial Intelligence (AI) that uses complex algorithms and precise curated evidence to calculate, propose, and help navigate a driver to customized target route options unique for each user and their given situation. In some embodiments, the system described herein is considered an AI solution where the data model is not driven by predictive machine learning (ML), but can be informed by ML. The scores themselves are neither direct predictions of efficacy nor per sent response or survival odds, though better matching has been associated with better clinical outcomes.

In some embodiments, an algorithm analyzes genomic information and other molecular data from the patient' cancer profile in the context of the expert-curated knowledge base of published pharmacological information, drug labels, clinical trials, and other evidence. From this information, the algorithm factors millions of potential drug combinations, and filters and computes scores for the resulting set of customized treatment options. This evidence-based reasoning approach is similar to that of a molecular tumor board and is the type of AI known as Knowledge Representation and Reasoning (KRR), which has been used on anything from scientific and clinical domains to robotics and navigation devices. It is also in the same category as expert rules-based AI tools widely used in medical systems. For clinical trial intelligence (CTI) analysis, each novel clinical trial drug, its therapy type, target(s), and the respective inhibitory concentration values (IC50), where applicable) are provided by the pharmaceutical company and added to the knowledgebase and preserved as a version or a special instance of the knowledge base.

In some embodiments, each matching option has interpretable Reasoning of the different pieces of Represented Knowledge connecting to the given molecular profile to justify the matching scores.

In some embodiments, molecular rule-based exclusions and warnings may be applied whenever certain seemingly matching therapies are filtered out due to risks of potential harms supported by evidence of toxicity, resistance, pharmacogenomics, other drug-drug interactions, or drug-marker incompatibilities.

In some embodiments, KRR AI does not depend on any prior dataset size, sampling, or outcomes data collection—factors critical for ML-driven approaches. As in the GPS Navigation Device analogy, the ability to map out possible routes for the target destinations does not depend on how many drivers previously took the same exact route at the same time of the day before and what outcomes this led to. The critical dependency is how precise the map content is and how many factors are taken into account to quickly personalize the calculations. However, KRR AI can incorporate additional knowledge and or algorithmic modifications from an ML type of model, which may uncover factors specific to a dataset with known molecular profiles, therapies given, and observed outcomes, such as functional studies and patient's survival and progression data.

In some embodiments, the matching scores are indicative of molecular matching degree, and the clinical validity of the matching score can be additionally established. The outcomes data from clinical trials shows a strong significant correlative relationship with patients' progression-free and overall survival (higher matching scores correlate with better PFS and or OS outcomes, and lower matching scores are predictive of poor clinical outcomes).

In some embodiments, the technical task solved by the methods disclosed herein is to help pharmaceutical companies bring better therapies to market in more time- and cost-efficient manner and thus improve clinical outcomes. The described technology, which is used for molecularly precise scoring and personalized selection of novel and known drug combinations and monotherapies can be applied to optimize and improve clinical trials. The system is used to empower physicians with augmented therapeutic intelligence solution aiding clinical decision making. The described methodology has been clinically validated to show that properly matched cancer therapies (with higher matching scores) are predictive of time to progression and overall survival (PFS and OS) of the patients. Poorly matched therapies are associated with more aggressive disease progression and poor overall survival outcomes in cancer patients irrespective of cancer types.

In some embodiments, the methods and systems disclosed herein can be applied to clinical patient stratification for better trial design to increase efficacy success, reduce adverse or toxic effects, dropouts, and trial failure. The disclosed methods and systems not only address the simple 1-to-1 match of biomarker to drug but can also propose and assess novel customized combinations using molecular basis for scoring, ranking, and filtering personalized precision therapeutics options.

In some embodiments, a machine learning technique can be used to determine or refine scoring thresholds for clinical trial patient stratification.

In some embodiments, the disclosed methods and systems utilize previously established knowledge base (database with curated clinical and scientific information or content), the platform that uses the database content, formulas, rules, and algorithms to generate, molecularly match, filter, score, and rank therapeutic options for any input patient's profile. The subsequent steps needed to inform clinical trials are: 1) utilizing matching scores for a given patient population and therapeutic considerations to determine numerical thresholds; and 2) stratifying patients into “enroll”, “do not enroll” and, optionally, “other” categories based on the threshold(s) determined in step (1).

In some embodiments, the disclosed methods and systems can be used to decipher how other molecular biomarkers can influence the proposed treatment's success or failure, alone or in various combinations, within a given population of patients. To maximize the population of patients that can benefit from novel therapies, eligibility can be further refined by applying the method to decipher molecular inclusion and exclusion criteria and or to alter therapy options considered in different arms of a given trial.

Some of the publications presenting clinical outcomes for the “matched” patients validate the customization of the best therapy, or best combination of drugs, in prospective clinical trial, including the following: Sicklick J K, et al., Molecular profiling of advanced malignancies guides first-line N-of-1 treatments in the I-PREDICT treatment-naïve study. Genome Med. 2021 Oct. 4; 13(1):155; Sicklick J K, et al., Molecular profiling of cancer patients enables personalized combination therapy: the I-PREDICT study. Nat Med. 2019 May; 25(5):744-750; Kato S, et al., Real-world data from a molecular tumor board demonstrates improved outcomes with a precision N-of-One strategy. Nat Commun. 2020 Oct. 2; 11(1):4965. The patients with higher molecular matching scores fared better than patients having lower molecular matching scores.

In some embodiments, certain aspects of generation of a matching score for the patient based on the molecular profile and the patient's predicted response to the therapy, as well as some aspects of the disclosed methods are disclosed in U.S. Pat. No. 11,434,534 B2, incorporated herein by reference.

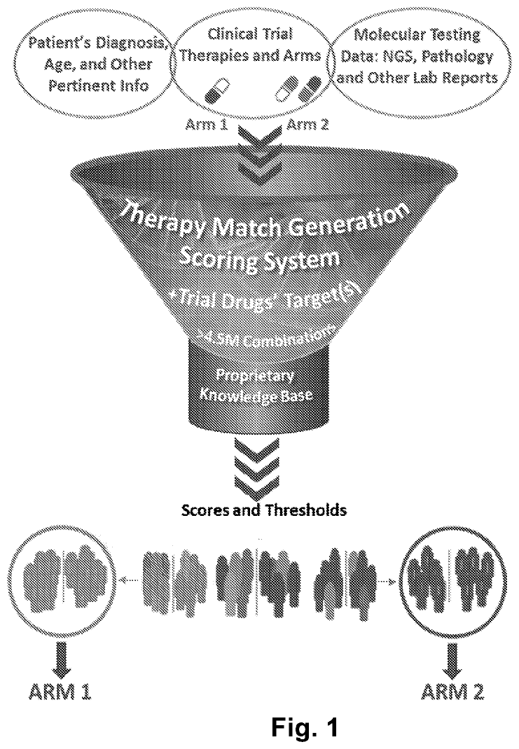

In some embodiments, the disclosed methods and systems provide patient stratification and can analyze multiple cohorts of data. The system can be used prospectively in trial design, or retrospectively for trial rescue. The system works on an individual patient, and also on a population level. This allows groups within the population to be identified and studied in a clinical trial. As shown in FIG. 1, this stratification can include decision support with respect to inclusion/exclusion of individual patients or groups of patients in a clinical trial, refine trial arm design, prevent toxicity, adverse events, and drop-outs.

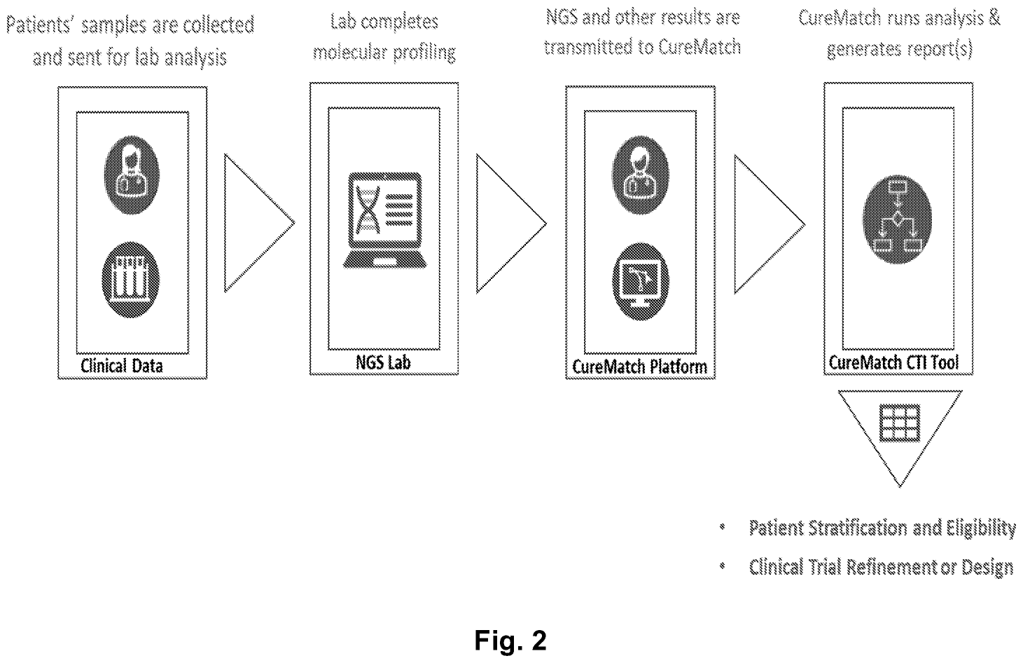

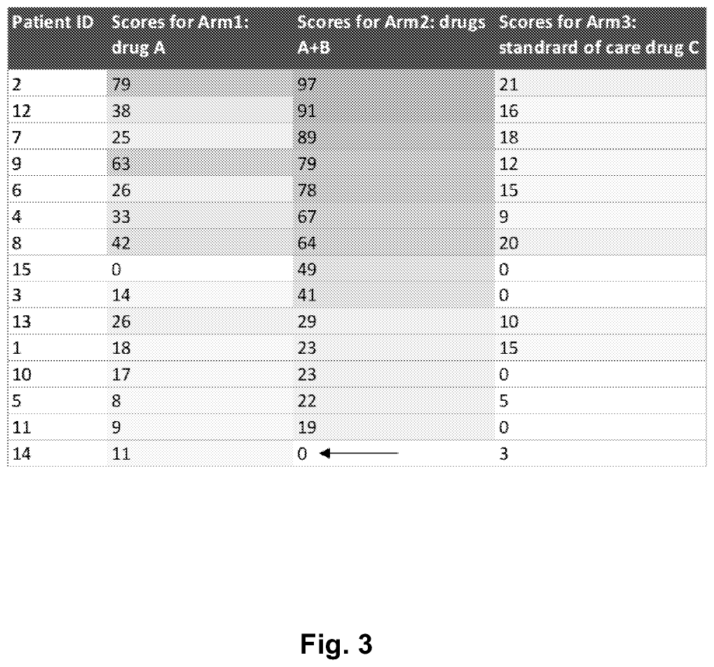

In some embodiments, the disclosed methods and systems can identify subpopulations of patients having different biomarkers. The system addresses and can refine arms of a clinical trial to avoid dropouts, prevent toxicity, etc. The clinical trial study can also be refined by creating additional arms, and providing additional options for drug and/or drug combinations in consideration for each arm. Real world data can be leveraged using this system to understand failure a clinical trial (see e.g., FIG. 2). In some embodiments, the entire distribution of scored drug or drug combination of interest applied to individual patients can be provided, and then a user can determines a threshold within the population for study in a clinical trial. For example, a user can determine which patients should be included or excluded from enrollment in a clinical trial. The disclosed system is agnostic to cancer type, sample type, panel, or platform.

FIG. 6 illustrates a user's data input. The figure shows the origin and the type of patient data that may be entered into Module 1 of a system. By way of example, a patient may visit a healthcare provider that records medical history and collects a sample suitable for molecular analysis. One or more samples from one or more tumor and or metastatic cancer sites are sent to a specialized laboratory to perform molecular profiling, including biomarker interpretation, where molecular types of data can be genomic/genetic (DNA), epigenetic (methylations status, other), transcriptomic (RNA), proteomic (protein), metabolomic, or other. Molecular results, clinical data, and user-defined parameters pertaining to any therapies of interest may be inputted in the system using a user-based interface.

Thus, in a first step, data may first be obtained. Using the example above, a patient may visit a physician, who obtains and records informative medical and demographics data (e.g. age, gender, histological diagnosis, previous treatment(s), current treatment, medical history and comorbidities, familial history and inherited syndromes, and the like) and performs a biopsy (tumor, blood, tissue of interest, body fluid, or the like) in order to define the status of biomarkers specific to the patient's disease. The status of biomarkers can be obtained from a validated third party company or an in-house laboratory, and can be assessed using methods of genomic profiling (e.g., DNA sequencing, comparative genomic hybridization, in situ hybridization or FISH, and the like), epigenomics profiling (e.g., histone modification assay, chromatin immunoprecipitation, restriction landmark analysis, bisulfite sequencing, and the like), transcriptomic profiling (e.g., microarray, RNA sequencing, real-time polymerase amplification, and the like), proteomic profiling (e.g., immunoassay, mass spectrometry, and the like), metabolomics profiling, or biochemistry profiling.

Next, data may be inputted. The physician (or designate) may log in to the system's web interface and enter information relevant to the system's use such as patient identification, patient diagnosis, patient treatment, and molecular description. At this stage, the physician may order a level of service depending of the type of results needed (e.g., de novo combination calculation or confirmation of a regimen already in use, consultation of previous results, level of confidence used to generate the results, etc.) and choose the maximum number of drugs to use.

In some embodiments, the disclosed method and system may comprise data processing, e.g., using a data-processing machine. In an aspect, after completion of a manual input of data, the system prepares the data for the machine analysis process. This step may comprise standardizing and categorizing the input data (e.g., medical, demographics, and molecular descriptions) in accordance to any existing controlled vocabulary and or ontologies used by the system (biomarker format, disease taxonomy, therapy types, targeting, and related indications.

In some embodiments, disclosed method and system incorporate the automated data input standardization. Data entered by the user may be checked for typographical errors or misspelling errors. All biologically relevant names (e.g., genes, proteins, alterations, pathology, and drugs) may be normalized using databases of synonyms where the system replaces input names with a unique reference name. A user can also perform additional steps relating to medical coding, medical billing, or configuration of the decision-making machine to a specific chosen level of service. A user can supply batch dataset information for multiple patients at once using tabular formats of data for one or more clinical cohort. Security features may be implemented in order to respect the confidentiality of protected health data and to ensure its safe usage of the protected health data throughout all steps in the process. The web interface may incorporate an automatic filling system, e.g., that points out any typographical errors or misspellings as stated above. Then, the analysis unit may query databases of synonyms to obtain unique names of genes, alterations, proteins, diagnosis, and drugs corresponding to the patient's description (e.g., when several terms exist for the same object). The system may replace all commonly used synonyms by a same, unique, reference name. For all of the following steps, the system may use but is not limited to the official gene names provided by the HUGO Gene Nomenclature Committee (HGNC), as well as world-wide used drug generic names. The standardization of a user's input may also take into account socioeconomic factors, such as medical codes (using ICD coding system) and medical billing information (depending on the level of services chosen and the health insurance details provided). Every component of the input standardization procedure and all subsequent procedures may respect the mandatory medical confidentiality requirements (e.g., Health Insurance Portability and Accountability Act (HIPAA) law in United States) and the entire system may have specific security features ensuring the safe use of protected health information (PHI).

In some embodiments, the disclosed method and system comprise the molecular description being transferred to a unit classifying the biomarkers' alterations by their functional effects. This module may interact with a database of gene functional effect information. This database may have a tiered structure. All data, regardless of tier, may be in the form of serialized data structures. Tiers may represent levels of significance or confidence and/or levels of service and the number of tiers can be changed according to the system's usage and requirements of specific analytical applications. Different functional effects may exist for the same alteration, reflecting the number of different data sources. A set of specific processing instructions may define a final unique conclusion to attribute to each alteration, taking into account the number of tier(s) to consider and the possible discrepancies existing between tiers. Only alterations considered functionally impacting the disease course or drug selection may be transmitted to the decision-making machine.

In some embodiments, the result of the input standardization may thus be transferred to a unit classifying the biomarkers' alterations by their functional effects as shown in the figure. This module may interact with a database of gene functional effect information, where the database may have a tiered structure, and where the data is in the form of serialized data structures. Each serialized structure may comprise the gene name or protein name, the specific description of the aberration (including, but not limited to, DNA level, RNA level, and protein level alterations), identifiers (including, but not limited to, genomic accession numbers and database identifiers) and a conclusion regarding the alteration's effect. The number of tiers can be modulated accordingly to the system's usage and may reflect different levels of significance and/or different levels of service. For example, in a model using three tiers, the first (lowest) tier may contain data with effect conclusions determined by basic analysis of protein regions and conserved sequences, e.g., extracted from public resources. The second tier may comprise data with effect conclusions determined through in vitro/in vivo experimentations, clinical observations, and published observations. The third tier may comprise data with effect conclusions determined by case studies and in-depth computer structure-activity modeling and simulations. There may be multiple categories with two or more possible tiers of content applied to each.

In some embodiments, different conclusions may exist for the same alteration, reflecting the number of data sources. A set of specific processing instructions may define a final unique conclusion for each alteration, taking into account all serialized conclusions and levels of significance or confidence known for said alteration. The nature of the final conclusions may depend on the disease studied and may dictate the relevance of the biomarker for the specific case. For example, in a case of cancer, the alteration effect may be summarized as ‘oncogenic,’ ‘non-oncogenic,’ ‘unknown,’ or ‘conflicting.’ Only alterations presenting an ‘oncogenic’ or ‘unknown’ functional effect may be kept for further consideration by the system (in this example). In all embodiments, the term ‘oncogenic’ may also imply ‘oncologic therapy-related’ and encompass any biomarkers, which are not disease-causing or disease-promoting themselves, yet, are known to be associated with sensitivity or resistance to any drug(s) targeting oncogenic processes.

In some embodiments, using the functional effects for each biomarker, the decision-making machine may apply specific processing instructions in order to define, and provide the user, the best combinations of available therapeutic options targeting as many biomarkers with a relevant effect on the disease progression as possible, preferably all.

In some embodiments, preferred therapies may comprise agents targeting the biomarker itself (“direct” targeting) or a component within the biomarker's canonical signaling pathway(s) (“semi-direct” or “indirect” targeting). For example, a drug “directly” inhibiting a ligand of a biomarker may be considered “semi-directly” targeting the biomarker, and a drug ‘directly’ targeting a molecule downstream of the biomarker's signaling pathway may be considered ‘indirectly’ targeting the biomarker.

In some embodiments, standardized input data may be used to determine all possible drugs for the given targets using information available in the accessible drugs and targeting relationships databases. These possible drugs may then be used with input parameters to generate every possible combination for the patient. The list of combinations may then be filtered for unacceptable conditions such as contraindications, redundant targeting, and toxicity, using specific functions and a set of appropriate databases. Retained (suitable) combinations may be scored, where the matching score of each combination is primarily based on the drugs' biological activity. The scored combinations may then be sorted by numerical scores and other desirable features such as indications and availability.

In some embodiments, the list of relevant biomarkers generated by the data-processing machine may be used to query a database of targeting relationships (‘direct,’ ‘semi-direct,’ or ‘indirect’) and attribute a specific way to target each alteration. The targeting relationships may be used to find corresponding drugs, e.g., using a database of available drugs, which may comprise local, United States, or foreign regulatory authorities' approved-drugs (e.g., Food and Drug Administration in United States, European Agency for Medicines in Europe, and so on), experimental drugs, or the user's self-designed compounds library.

In some embodiments, a list of optimized therapies may be generated based on available patient's information. This may include combining available and suitable drugs in order to produce therapeutic regimens acting on one or several molecular alterations presented by the patient. These combinations may be generated by different methods. For example, combinations may be built by selecting at least one drug for each existing target, leading to a complete coverage of all molecular alterations (but potentially increasing the toxicity through the high number of drugs used), or by defining a maximum number of drugs to combine, leading to a lower level of toxicity but potentially presenting a lack of efficiency due to an incomplete coverage of molecular alterations.

FIG. 7 illustrates a computer system. The computer system 1200 may be part of, or otherwise implemented with, the environment for personalized medicine, e.g., described with reference to FIG. 1. For example, the computer system 1200 may be the data processing machine, the decision making machine, the learning machines, and the like described with reference to FIG. 1, or the computer system 1200 may otherwise be comprised as part of any of those machines. In an aspect, a computing device 1210 of the computer system 1200 is a network device operated by one or more users (e.g., physician or patient) in the system shown in FIG. 1. In certain aspects, the computer system 1200 may be implemented using hardware or a combination of software and hardware, either in a dedicated server, or integrated into another entity, or distributed across multiple entities. In general, the computer system 1200 may comprise a computing device 1210 connected to a network 1202, e.g., through an external device 1204.

The computing device 1210 may comprise a desktop computer workstation. The computing device 1210 may also or instead be any device suitable for interacting with other devices over a network 1202, such as a laptop computer, a desktop computer, a personal digital assistant, a tablet, a mobile phone, a television, a set top box, a wearable computer, and the like. The computing device 1210 may also or instead comprise a server or it may be disposed on a server, such as any of the servers described herein.

The computing device 1210 may be used for any of the entities described in the personalized medicine techniques and systems, e.g., as described above with reference to FIG. 1. In certain aspects, the computing device 1210 may be implemented using hardware (e.g., in a desktop computer), software (e.g., in a virtual machine or the like), or a combination of software and hardware. The computing device 1210 may be a standalone device, a device integrated into another entity or device, a platform distributed across multiple entities, or a virtualized device executing in a virtualization environment.

The network 1202 may comprise any network described above, e.g., data network(s) or internetwork(s) suitable for communicating data and control information among participants in the computer system 1200. This may comprise public networks such as the Internet, private networks, and telecommunications networks such as the Public Switched Telephone Network or cellular networks using third generation cellular technology (e.g., 3G or IMT-2000), fourth generation cellular technology (e.g., 4G, LTE. MT-Advanced, E-UTRA, etc.) or WiMax-Advanced (IEEE 802.16m)) and/or other technologies, as well as any of a variety of corporate area, metropolitan area, campus or other local area networks or enterprise networks, along with any switches, routers, hubs, gateways, and the like that might be used to carry data among participants in the computer system 1200. The network 1202 may also comprise a combination of data networks, and need not be limited to a strictly public or private network.

The external device 1204 may be any computer or other remote resource that connects to the computing device 1210 through the network 1202. This may comprise personalized medicine resources such as any of those contemplated herein, gateways or other network devices, remote servers or the like containing content requested by the computing device 1210, a network storage device or resource, a device hosting personalized medicine content or data, or any other resource or device that might connect to the computing device 1210 through the network 1202.

In another aspect, the external device 1204 is a server, where the computing device 1210 is a rack within the server. Such a server may comprise multiple such racks. Also, various servers, which may act in concert to perform processes described herein, may be disposed in different geographic locations. The servers may coordinate their operation in order to provide the capabilities to implement processes described herein. The servers may provide interfaces to other devices including, without limitation, clients, other servers, printers, database servers, print servers, file servers, communication servers, distributed servers, and the like, where any and all of which may be comprised as another external device 1204 in the computer system 1200. The processes and techniques described herein may be implemented on one such server or on multiple such servers.

In general, the computing device 1210 may comprise a processor 1212, a memory 1214, a network interface 1216, a data store 1218, and one or more input/output interfaces 1220. The computing device 1210 may further comprise or be in communication with peripherals 1222 and other external input/output devices that might connect to the input/output interfaces 1220.

The processor 1212 may be any processor or other processing circuitry capable of processing instructions for execution within the computing device 1210 or computer system 1200. The processor 1212 may comprise a single-threaded processor, a multi-threaded processor, a multi-core processor and so forth. The processor 1212 may be capable of processing instructions stored in the memory 1214 or the data store 1218.

The memory 1214 may store information within the computing device 1210. The memory 1214 may comprise any volatile or non-volatile memory or other computer-readable medium, including without limitation a Random Access Memory (RAM), a flash memory, a Read Only Memory (ROM), a Programmable Read-only Memory (PROM), an Erasable PROM (EPROM), registers, and so forth. The memory 1214 may store program instructions, program data, executables, and other software and data useful for controlling operation of the computing device 1210 and configuring the computing device 1210 to perform functions for a user. The memory 1214 may comprise a number of different stages and types of memory for different aspects of operation of the computing device 1210. For example, a processor may comprise on-board memory and/or cache for faster access to certain data or instructions, and a separate, main memory or the like may be comprised to expand memory capacity as desired. All such memory types may be a part of the memory 1214 as contemplated herein.

The memory 1214 may, in general, comprise a non-volatile computer readable medium containing computer code that, when executed by the computing device 1210 creates an execution environment for a computer program in question, e.g., code that constitutes processor firmware, a protocol stack, a database management system, an operating system, or a combination of the foregoing, and/or code that performs some or all of the steps set forth in the various flow charts and other algorithmic descriptions set forth herein. While a single memory 1214 is depicted, it will be understood that any number of memories may be usefully incorporated into the computing device 1210. For example, a first memory may provide non-volatile storage such as a disk drive for permanent or long-term storage of files and code even when the computing device 1210 is powered down. A second memory such as a random access memory may provide volatile (but higher speed) memory for storing instructions and data for executing processes. A third memory may be used to improve performance by providing higher speed memory physically adjacent to the processor 1212 for registers, caching, and so forth. The processor 212 and the memory 214 can be supplemented by, or incorporated in, logic circuitry.

The network interface 1216 may comprise any hardware and/or software for connecting the computing device 1210 in a communicating relationship with other resources through the network 1202. This may comprise remote resources accessible through the Internet, as well as local resources available using short range communications protocols using, e.g., physical connections (e.g., Ethernet), radio frequency communications (e.g., WiFi), optical communications, (e.g., fiber optics, infrared, or the like), ultrasonic communications, or any combination of these or other media that might be used to carry data between the computing device 1210 and other devices. The network interface 1216 may, for example, comprise a router, a modem, a network card, an infrared transceiver, a radio frequency (RF) transceiver, a near field communications interface, a radio-frequency identification (RFID) tag reader, or any other data reading or writing resource or the like.

More generally, the network interface 1216 may comprise any combination of hardware and software suitable for coupling the components of the computing device 1210 to other computing or communications resources. By way of example and not limitation, this may comprise electronics for a wired or wireless Ethernet connection operating according to the IEEE 802.11 standard (or any variation thereof), or any other short or long range wireless networking components or the like. This may comprise hardware for short range data communications such as Bluetooth or an infrared transceiver, which may be used to couple to other local devices, or to connect to a local area network or the like that is in turn coupled to a data network 1202 such as the Internet. This may also or instead comprise hardware/software for a WiMax connection or a cellular network connection (using, e.g., CDMA, GSM, LTE, or any other suitable protocol or combination of protocols). The network interface 1216 may be comprised as part of the input/output devices 1220 or vice-versa.

The data store 1218 may be any internal memory store providing a computer-readable medium such as a disk drive, an optical drive, a magnetic drive, a flash drive, or other device capable of providing mass storage for the computing device 1210. The data store 1218 may store computer readable instructions, data structures, program modules, and other data for the computing device 1210 or computer system 1200 in a non-volatile form for subsequent retrieval and use. For example, the data store 1218 may store without limitation one or more of the operating system, application programs, program data, databases, files, and other program modules or other software objects and the like.

The input/output interface 1220 may support input from and output to other devices that might couple to the computing device 1210. This may, for example, comprise serial ports (e.g., RS-232 ports), universal serial bus (USB) ports, optical ports, Ethernet ports, telephone ports, audio jacks, component audio/video inputs, HDMI ports, and so forth, any of which might be used to form wired connections to other local devices. This may also or instead comprise an infrared interface, RF interface, magnetic card reader, or other input/output system for coupling in a communicating relationship with other local devices. It will be understood that, while the network interface 1216 for network communications is described separately from the input/output interface 1220 for local device communications, these two interfaces may be the same, or may share functionality, such as where a USB port is used to attach to a WiFi accessory, or where an Ethernet connection is used to couple to a local network attached storage.

A peripheral 1222 may comprise any device used to provide information to or receive information from the computing device 1200. This may comprise human input/output (I/O) devices such as a keyboard, a mouse, a mouse pad, a track ball, a joystick, a microphone, a foot pedal, a camera, a touch screen, a scanner, or other device that might be employed by the user 1230 to provide input to the computing device 1210. This may also or instead comprise a display, a speaker, a printer, a projector, a headset or any other audiovisual device for presenting information to a user. The peripheral 1222 may also or instead comprise a digital signal processing device, an actuator, or other device to support control or communication to other devices or components. Other I/O devices suitable for use as a peripheral 1222 comprise haptic devices, three-dimensional rendering systems, augmented-reality displays, magnetic card readers, and so forth. In one aspect, the peripheral 1222 may serve as the network interface 1216, such as with a USB device configured to provide communications via short range (e.g., BlueTooth, WiFi, Infrared, RF, or the like) or long range (e.g., cellular data or WiMax) communications protocols. In another aspect, the peripheral 1222 may provide a device to augment operation of the computing device 1210, such as a global positioning system (GPS) device, a security dongle, or the like. In another aspect, the peripheral may be a storage device such as a flash card, USB drive, or other solid state device, or an optical drive, a magnetic drive, a disk drive, or other device or combination of devices suitable for bulk storage. More generally, any device or combination of devices suitable for use with the computing device 1200 may be used as a peripheral 1222 as contemplated herein.

Other hardware 1226 may be incorporated into the computing device 1200 such as a co-processor, a digital signal processing system, a math co-processor, a graphics engine, a video driver, and so forth. The other hardware 1226 may also or instead comprise expanded input/output ports, extra memory, additional drives (e.g., a DVD drive or other accessory), and so forth.

A bus 1232 or combination of busses may serve as an electromechanical platform for interconnecting components of the computing device 1200 such as the processor 1212, memory 1214, network interface 1216, other hardware 1226, data store 1218, and input/output interface. As shown in the figure, each of the components of the computing device 1210 may be interconnected using a system bus 1232 or other communication mechanism for communicating information.

Methods and systems described herein may be realized using the processor 1212 of the computer system 1200 to execute one or more sequences of instructions contained in the memory 1214 to perform predetermined tasks. In embodiments, the computing device 1200 may be deployed as a number of parallel processors synchronized to execute code together for improved performance, or the computing device 1200 may be realized in a virtualized environment where software on a hypervisor or other virtualization management facility emulates components of the computing device 1200 as appropriate to reproduce some or all of the functions of a hardware instantiation of the computing device 1200.

The coupling and/or connection between components in the computer system 1200, such as those described above, may facilitate remote execution of programs across the network 1202. In this manner, the networking of some or all of these devices may facilitate parallel processing of a program or a method at one or more location without deviating from the scope of the implementations described herein. In addition, any of the devices attached to components in the computer system 1200 (e.g., a server) through an interface may comprise at least one storage medium capable of storing methods, programs, code and/or instructions. A central repository may provide program instructions to be executed on different devices. In such an implementation, the remote repository may act as a storage medium for program code, instructions, and programs.

Implementations described herein can be implemented using a computer system 200 in response to the processor 1212 executing one or more sequences of one or more instructions contained in the memory 1214. Such instructions may be read into the memory 1214 from another machine-readable medium, such as the data store 1218. Execution of the sequences of instructions contained in the memory 1214 may cause the processor 1212 to perform processes described herein. One or more processors 1212 in a multi-processing arrangement may also be employed to execute the sequences of instructions contained in the memory 1214. In alternative aspects, hard-wired circuitry may be used in place of or in combination with software instructions to implement various aspects of the present disclosure. Thus, aspects of the present disclosure are not limited to any specific combination of hardware circuitry and software.

### Cloud Implementation

The proposed system can be implemented to run in one of the computational environments offered by a cloud provider. Such implementation can fully utilize low to medium level interfaces accessible by the underlying layers to interact with users, process input requests, store and retrieve necessary data. It accommodates all input-output processing and calculation requiring only business logic implementation and not the standard system routines, such as serving various standard protocols.

In the proposed solution, Java computer language was chosen to better utilize the existing enterprise frameworks to focus on high availability and the ease of code implementation, support and deployment in the modern heterogeneous computational infrastructures. All modules and interfaces, including user-interface of the system are bundled together by the Jenkins build system as Enterprise Application and archived for ease of deployment.

For example in the FIG. 8, the block diagram illustrates the implemented application deployed into the configured enterprise container on one of the Elastic Compute Cloud (EC2) Virtual Machine (VM) instances configured in the cloud solution (e.g. Amazon Web Services, Google Cloud . . . ). In order to address fault-tolerance demands, more than one computational instances proposed for production deployment may be used. Such virtual instances must be identically configured to run the Linux operating system with the same number of available central processing unit (CPU) cores, memory and hard drive capacity. Each instance has a configured enterprise Java server container running to serve the application for the proposed system with high availability. In this example the most up-to-date Apache Tomcat 8.0 container and Java JDK 8 are used.

Only one application server on the primary instance is serving requests to the application via the load balancer to guarantee that service is provided at all times. In case of failure in one or more instances, requests will be routed to the back-up fail-over instance. The cloud provider guarantees high availability by scaling the number of configured primary instances to address high volumes of usage and recovery procedures for failed instances.

The implemented application communicates with a database configured in the cluster for storing and retrieving necessary data. Any Structured Query Language (SQL) compatible with SQL92 relational database services supporting the Java Database Connectivity (JDBC) protocol for communication such as Microsoft SQL Server, Oracle, PostgreSQL or MySQL can be used. In this example we rely on Aurora SQL database provided by Amazon Web Services in the Amazon Relational Database Service (RDS) environment. This Aurora database server is similar to the MySQL database server, but oriented towards ease of setup and deployment in a fault-tolerant cloud-based cluster environment. In the provided example, the database cluster utilizes at least two instances running the Linux Operating System and running database services with data replication from primary to fail-over nodes to provide uninterrupted database access. For high availability multiple primary nodes can be added to address high volumes of requests. The data storage is encrypted to provide increased security.

In the provided example, the application utilizes file storage buckets in the Amazon Simple Storage Service (S3) environment for secure access to large amounts of data required for processing by various subsystems of Application of proposed system.

All the communication within the cloud infrastructure between EC2, S3 and RDS is performed securely to protect all vital information within an isolated “secure group”. End-users and system supervisors, including experts access the system via web-based interface and provide all the necessary input information. In the proposed example Hyper Text Transport Protocol Secure (HTTPS) protocol is used to protect all inbound and outbound communication with the Load Balancer configuration.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings.

The systems and methods disclosed herein may be implemented via one or more components, systems, servers, appliances, other subcomponents, or distributed between such elements. When implemented as a system, such systems may comprise an/or involve, inter alia, components such as software modules, general-purpose CPU, RAM, etc., found in general-purpose computers. In implementations where the innovations reside on a server, such a server may comprise or involve components such as CPU, RAM, etc., such as those found in general-purpose computers.

Additionally, the systems and methods herein may be achieved via implementations with disparate or entirely different software, hardware and/or firmware components, beyond that set forth above. With regard to such other components (e.g., software, processing components, etc.) and/or computer-readable media associated with or embodying the present implementations, for example, aspects of the innovations herein may be implemented consistent with numerous general purpose or special purpose computing systems or configurations. Various exemplary computing systems, environments, and/or configurations that may be suitable for use with the innovations herein may comprise, but are not limited to: software or other components within or embodied on personal computers, servers or server computing devices such as routing/connectivity components, hand-held or laptop devices, multiprocessor systems, microprocessor-based systems, set top boxes, consumer electronic devices, network PCs, other existing computer platforms, distributed computing environments that comprise one or more of the above systems or devices, etc.

In some instances, aspects of the systems and methods may be achieved via or performed by logic and/or logic instructions including program modules, executed in association with such components or circuitry, for example. In general, program modules may comprise routines, programs, objects, components, data structures, etc., that perform particular tasks or implement particular instructions herein. The embodiments may also be practiced in the context of distributed software, computer, or circuit settings where circuitry is connected via communication buses, circuitry or links. In distributed settings, control/instructions may occur from both local and remote computer storage media including memory storage devices.

The software, circuitry and components herein may also comprise and/or utilize one or more type of computer readable media. Computer readable media can be any available media that is resident on, associable with, or can be accessed by such circuits and/or computing components. By way of example, and not limitation, computer readable media may comprise computer storage media and communication media. Computer storage media comprises volatile and nonvolatile, removable and non-removable media implemented in any method or technology for storage of information such as computer readable instructions, data structures, program modules or other data. Computer storage media comprises, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Further, communication media may comprise wired media such as a wired network or direct-wired connection, where media of any type herein does not comprise transitory media. Combinations of the any of the above are also comprised within the scope of computer readable media.

In the present description, the terms component, module, device, etc. may refer to any type of logical or functional software elements, circuits, blocks and/or processes that may be implemented in a variety of ways. For example, the functions of various circuits and/or blocks can be combined with one another into any other number of modules. Each module may even be implemented as a software program stored on a tangible memory (e.g., random access memory, read only memory, CD-ROM memory, hard disk drive, etc.) to be read by a central processing unit to implement the functions of the innovations herein. Or, the modules can comprise programming instructions transmitted to a general purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays or any mix thereof which provides the desired level performance and cost.

As disclosed herein, features consistent with the disclosure may be implemented via computer-hardware, software and/or firmware. For example, the systems and methods disclosed herein may be embodied in various forms including, for example, a data processor, such as a computer that also comprises a database, digital electronic circuitry, firmware, software, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the implementations described herein or they may comprise a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the implementations herein, or it may be more convenient to construct a specialized apparatus or system to perform the required methods and techniques.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects comprise: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied comprise, but are not limited to, non-volatile storage media in various forms (e.g., optical, magnetic or semiconductor storage media) though again does not comprise transitory media. Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Additionally, the words “herein,” “hereunder,” “above,” “below,” and words of similar import refer to this application as a whole and not to any particular portions of this application.

Moreover, the above systems, devices, methods, processes, and the like may be realized in hardware, software, or any combination of these suitable for a particular application. The hardware may comprise a general-purpose computer and/or dedicated computing device. This comprises realization in one or more microprocessors, microcontrollers, embedded microcontrollers, programmable digital signal processors or other programmable devices or processing circuitry, along with internal and/or external memory. This may also, or instead, comprise one or more application specific integrated circuits, programmable gate arrays, programmable array logic components, or any other device or devices that may be configured to process electronic signals. It will further be appreciated that a realization of the processes or devices described above may comprise computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software. In another aspect, the methods may be embodied in systems that perform the steps thereof, and may be distributed across devices in a number of ways. At the same time, processing may be distributed across devices such as the various systems described above, or all of the functionality may be integrated into a dedicated, standalone device or other hardware. In another aspect, means for performing the steps associated with the processes described above may comprise any of the hardware and/or software described above. All such permutations and combinations are intended to fall within the scope of the present disclosure.

Embodiments disclosed herein may comprise computer program products comprising computer-executable code or computer-usable code that, when executing on one or more computing devices, performs any and/or all of the steps thereof. The code may be stored in a non-transitory fashion in a computer memory, which may be a memory from which the program executes (such as random access memory associated with a processor), or a storage device such as a disk drive, flash memory or any other optical, electromagnetic, magnetic, infrared or other device or combination of devices. In another aspect, any of the systems and methods described above may be embodied in any suitable transmission or propagation medium carrying computer-executable code and/or any inputs or outputs from same.

It will be appreciated that the devices, systems, and methods described above are set forth by way of example and not of limitation. Absent an explicit indication to the contrary, the disclosed steps may be modified, supplemented, omitted, and/or re-ordered without departing from the scope of this disclosure. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context.

The method steps of the implementations described herein are intended to comprise any suitable method of causing such method steps to be performed, consistent with the patentability of the following claims, unless a different meaning is expressly provided or otherwise clear from the context. So for example performing the step of X comprises any suitable method for causing another party such as a remote user, a remote processing resource (e.g., a server or cloud computer) or a machine to perform the step of X. Similarly, performing steps X, Y and Z may comprise any method of directing or controlling any combination of such other individuals or resources to perform steps X, Y and Z to obtain the benefit of such steps. Thus method steps of the implementations described herein are intended to comprise any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

The following enumerated embodiments are representative of the invention:
1. A method for assessing eligibility of a patient to a clinical trial of a therapy comprising one or more drugs, the method comprising:
(a) providing at least one drug target for the one or more drugs, and providing a molecular profile for the patient, wherein the molecular profile is generated from analysis of a biological sample obtained from the patient and contains one or more clinically relevant biomarkers;
(b) generating a matching score for the patient based on the molecular profile and the patient's predicted response to the therapy, and setting a threshold score, wherein the generating step comprises determining impact of the therapy on the molecular profile of the patient based on the number and types of biomarkers and interactions between biomarkers and the one or more drugs of the therapy;
(c) providing a recommendation for clinical trial eligibility of the patient when the matching score for the patient passes requirements of the threshold score.
2. The method of embodiment 1, wherein eligibilities of a plurality of patients are assessed, and the matching score is generated for each patient of the plurality of patients.
3. The method of embodiment 2, wherein the threshold score is set after the matching score for each patient of the plurality of patients is generated.
4. The method of embodiment 3, wherein the threshold score is set at a specific percentile of a range of matching scores generated for each patient of the plurality of patients.
5. The method of embodiment 2, wherein the threshold score is set before the matching score for each patient of the plurality of patients is generated.
6. The method of any one of embodiments 1-5, wherein the threshold score is set based on previously analyzed clinical trial data sets.
7. The method of any one of embodiments 1-6, wherein the threshold score is set based on a newly obtained patient data from the clinical trial.
8. The method of any one of embodiments 1-7, wherein the providing at least one drug target for the drug comprises providing an IC50 value of the drug for the at least one drug target.
9. The method of any one of embodiments 1-8, wherein the patient is a cancer patient.
10. The method of any one of embodiments 1-9, wherein the biological sample comprises a tissue, blood, plasma or serum sample.
11. The method of any one of embodiments 1-10, wherein the analysis of the biological sample comprises performing a next-generation sequencing of nucleic acid molecules extracted from the biological sample.
12. The method of embodiment 11, wherein the analysis of the biological sample further comprises extracting a cell-free DNA from the biological sample.
13. The method of any one of embodiments 1-12, wherein the clinical trial is a prospective clinical trial.
14. The method of any one of embodiments 1-13, wherein generating the matching score further comprising determining impact of the drug on each of the one or more clinically relevant biomarkers.
15. The method of any one of embodiments 1-14, wherein the one or more clinically relevant biomarkers is/are targeted by the one or more drugs of the therapy.
16. A system for assessing eligibility of a patient to a clinical trial of a drug, the system comprising:
a server;
a computing device in communication with the server over a network, the computing device including a processor and a memory, the memory bearing computer executable code configured to perform the steps of:
(a) providing at least one drug target for the one or more drugs, and providing a molecular profile for the patient, wherein the molecular profile is generated from analysis of a biological sample obtained from the patient and contains one or more clinically relevant biomarkers;
(b) generating a matching score for the patient based on the molecular profile and the patient's predicted response to the therapy, and setting a threshold score, wherein the generating step comprises determining impact of the therapy on the molecular profile of the patient based on the number and types of biomarkers and interactions between biomarkers and the one or more drugs of the therapy;
(c) providing a recommendation for clinical trial eligibility of the patient when the matching score for the patient passes requirements of the threshold score.
17. The system of embodiment 16, wherein eligibilities of a plurality of patients are assessed, and the matching score is generated for each patient of the plurality of patients.
18. The system of embodiment 17, wherein the threshold score is set after the matching score for each patient of the plurality of patients is generated.
19. The system of embodiment 17, wherein the threshold score is set at a specific percentile of a range of matching scores generated for each patient of the plurality of patients.
20. The system of any one of embodiments 16-19, wherein the one or more clinically relevant biomarkers is/are targeted by the one or more drugs of the therapy.
21. The system of any one of embodiments 16-20, wherein the patient is a cancer patient.
22. The system of any one of embodiments 16-21, wherein the biological sample comprises a tissue, blood, plasma or serum sample.
23. The system of any one of embodiments 16-22, wherein generating the matching score further comprising determining impact of the drug on each of the one or more clinically relevant biomarkers

### EXAMPLES

Aspects of the present teachings may be further understood in light of the following examples, which should not be construed as limiting the scope of the present teachings in any way.

### Example 1

In this example, considerations for determining clinical trial eligibility for patients diagnosed with a proliferative, degenerative or inflammatory disease are discussed, where therapy comprises an immunotherapy.

It was investigated (see e.g., US 20200362418 A1, incorporated herein by refererence) that AID/APOBEC mutational signature is associated with a better outcome following treatment by PD-1/PD-L1 blockade. At the same time, AID/APOBEC mutational signature is associated with an increase of neo-peptide hydrophobicity and PD-L1 mRNA expression in a large collection of human tumor samples. This was determined by investigating the molecular profile (point mutations and small insertions/deletions and mRNA expression data obtained by next-generation sequencing (NGS) methods) of 469 highly-mutated pan-cancer human tumors, available without restriction of use from the community resource project The Cancer Genome Atlas (TCGA) (Broad GDAC Firehose website).

Using the mutation description available for these tumors, all possible 8-mer to 10-mer neo-peptides were generated encompassing each mutation (n=2,660,232 epitopes located in 15,163 different gene products). The differences in total hydrophobicity (i.e. the sum of hydrophobicity of all residues) of the neo-peptides after versus before mutagenesis were then considered. The results obtained were computed in two ways-either not weighted by mRNA expression levels or weighted by these levels (in order to take into consideration whether the neo-antigens were actually transcribed and their respective levels of expression). Finally, the hydrophobicity and expression of immune markers of tumors harboring an AID/APOBEC mutational signature were compared to those without, using a Wilcoxon-Mann-Whitney rank-sum test and a Fisher's exact test, respectively. As a result, highly mutated tumors (top 30% tumor mutation burden in the TCGA database) presenting an AID/APOBEC mutational signature presented a significant increase in terms of overall change in hydrophobicity in comparison to tumors not altered by the AID/APOBEC enzymes (mean [confidence interval 95% (CI₉₅%)]=8,702 [7,506-9,898] versus 3,374 [2,987-3,761] arbitrary units (AU)—p-value<0.0001).

Accordingly, when the therapy investigated in the clinical trial comprises an immunotherapy, molecular profile for a patient generated from analysis of patient's biological sample may comprise a set of genomic and/or protein alterations obtained from the sample. Next, elucidating possible 8- to 10-mer peptides encompassing the set of genomic and/or protein alterations will result in a set of neo-epitopes present in the patient's sample. Antigenicity and immunogenicity of the set of neo-epitopes can be estimated based on determining physicochemical properties of neo-epitopes, including hydrophobicity, from the set of neo-epitopes as compared to corresponding epitopes that are normally present in a healthy/non-mutated cell. Further, the antigenicity and immunogenicity estimates can be used as biomarkers for prediction of the patient's response to immunotherapy.

### Example 2

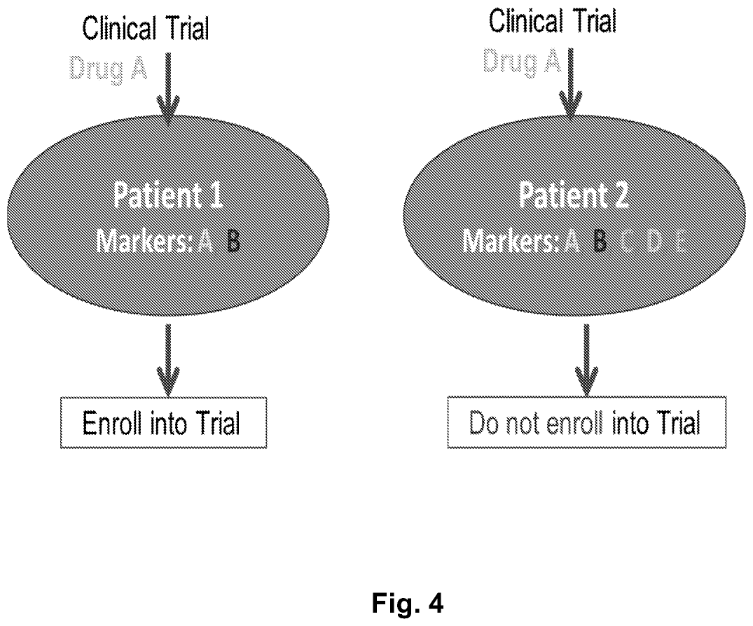

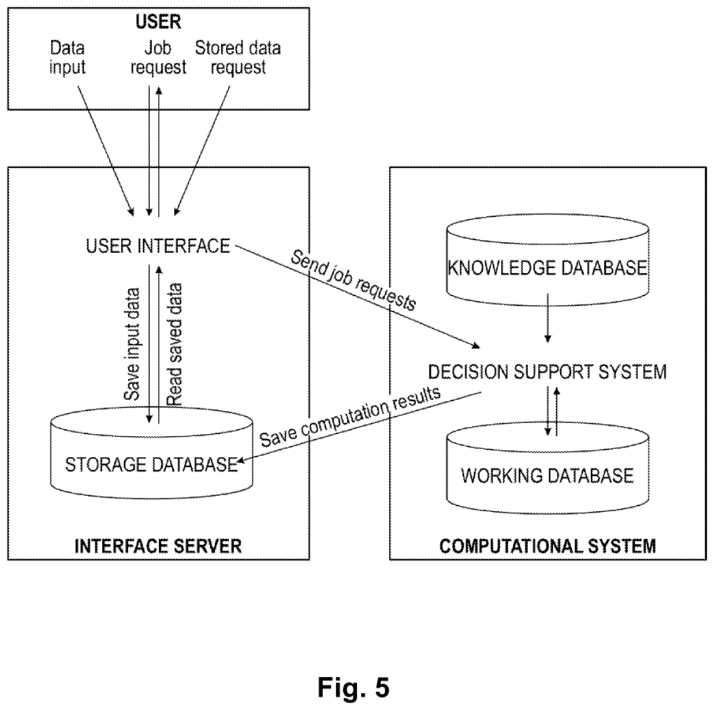

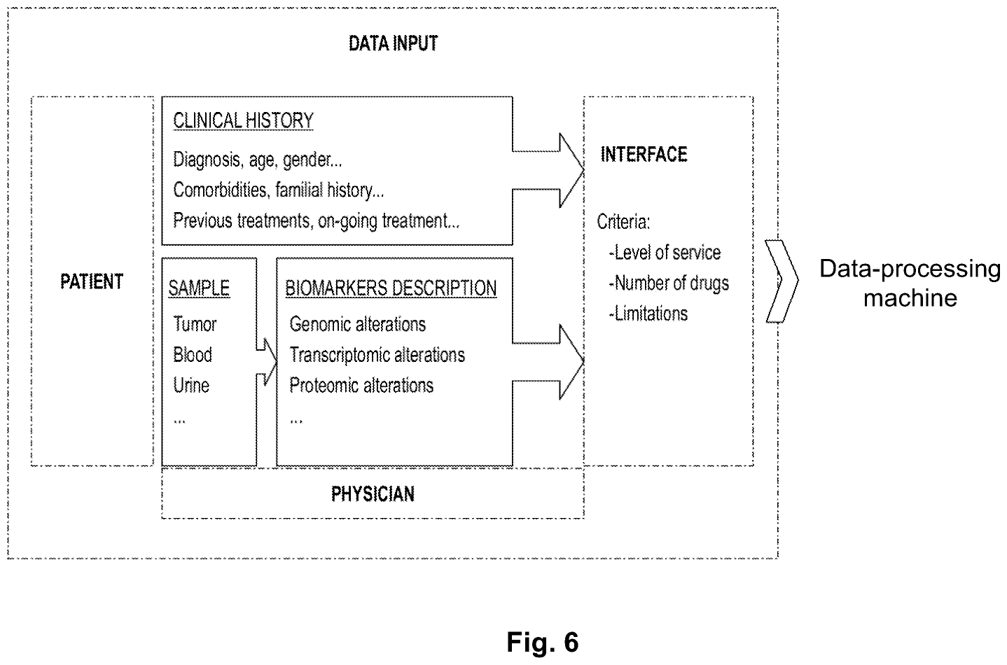

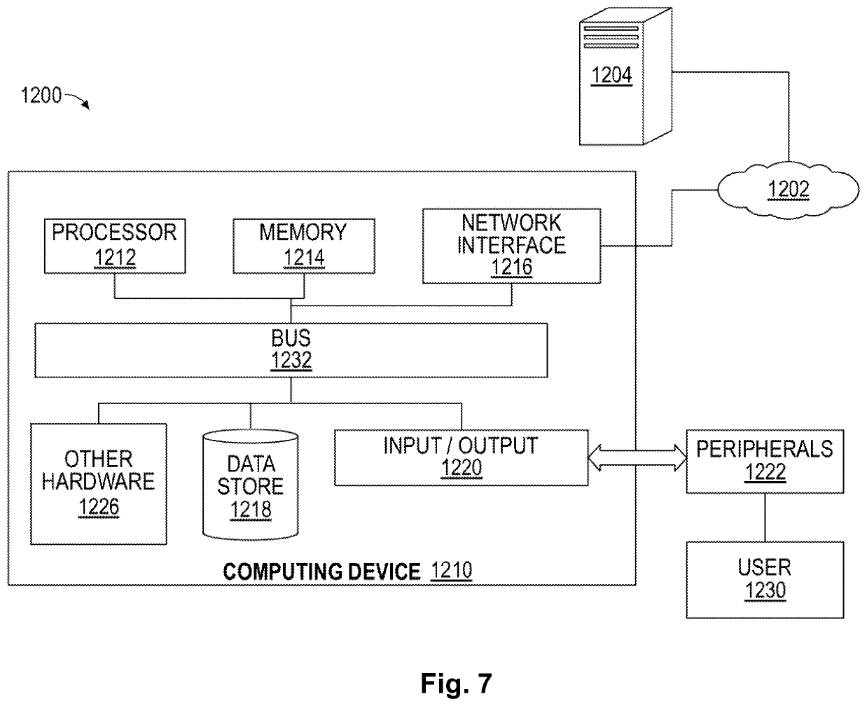

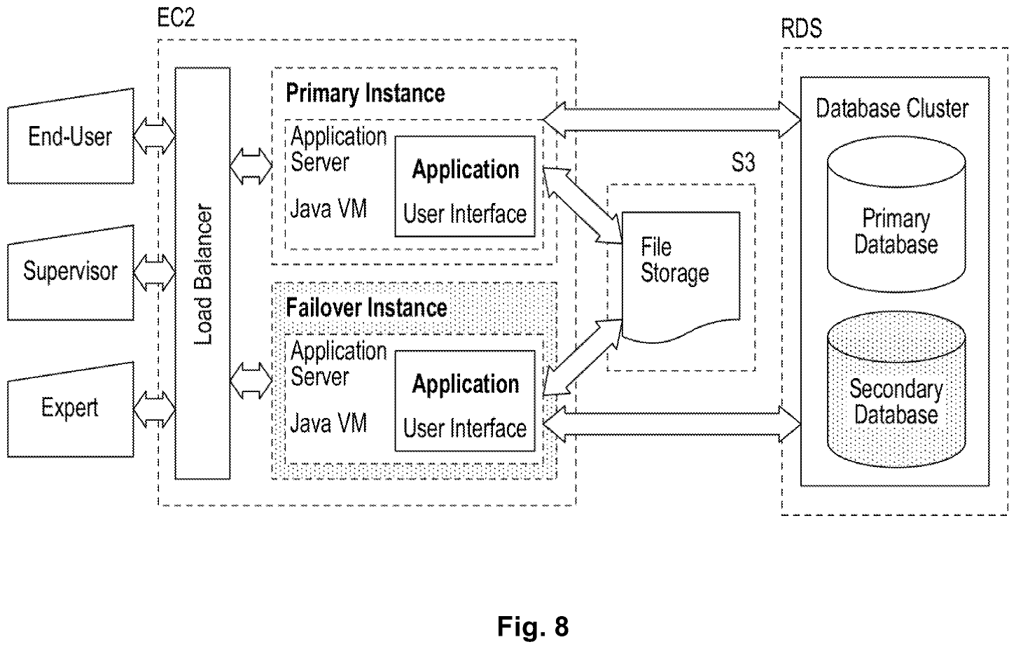

In this example, generation of a matching score based on patients' biomarkers and interactions between biomarkers and the one or more drugs of the therapy are discussed. FIG. 4 illustrates a general outcome for disclosed methods to exemplary patients: patient 1 receives a recommendation for clinical trial eligibility based on his/her molecular profile (biomarkers), while patient 2 receives a “no-go” (non-eligibility) recommendation, or no recommendation at all. The following scenarios illustrate how to study a population of patients in a clinical trial, and determine which patient will respond best to a drug or combination of drugs. The system can analyze the molecular profile of every tumor in every patient in a clinical trial to better predict which group of patients in the overall population will respond best (or worst) to the drug of interest.
A. Patient N1. In a trial investigating a novel direct KRAS (exclusively) inhibitor (Drug A), a patient presents with a KRAS DNA gain-of-function/activating alteration that is biologically and clinically relevant and known to cause or contribute to disease. No other clinically relevant biomarkers are detected from NGS and or other molecular profiling tests. The score for this patient for Drug A would be approaching ˜100%, depending on the specific IC50 value provided.
B. Patient N2. In the same trial, another patient harbors the same marker as above and another alteration—a genomic fusion, which is a known pathogenic biomarker that is not considered targetable by any existing drugs. The score would be at best near 50%.
C. Patient N3. In the same trial, a patient has the same KRAS biomarker and another marker that is not targetable, similar to Patient 2. Additionally, Patient 3 presents with 3 other pathogenic markers in known cancer-related genes EGFR, KRAS, PTEN. Since there is an un-targetable biomarker and higher molecular complexity (5 markers overall), the score for the KRAS inhibiting trial drug alone would be <20%, and the best possible score for combination therapies (if such were considered in any of the trial's arms) may be >70%.
D. Patient N4. In a clinical trial evaluating a novel check-point inhibitor immunotherapy monoclonal antibody drug, a patient presents with high PD-L1 expression and a high tumor mutation burden (TMB). The score for this drug for the given patient would be ˜100%.
E. Patient N5. In the same trial as Patient 4, a patient presents with the same PD-L1 and TMB levels, but also harbors 2 other biomarkers, one of which is EGFR amplification (high copy number gain). The score for the same drug may be 0%, because immune checkpoint inhibitors are excluded as therapy options in EGFR-mutant cancer, unless EGFR in most cases due to the risk of hyperprogression [PMID: 28351930, PMID: 34113560].
F. Patient N6. A patient with a KRAS gain-of-function mutation and a PTEN loss is considered for a trial evaluating a novel mTOR-specific inhibitor (a known cancer drug target used to address PTEN loss upstream in the Pi3k/mTOR pathway). The score would be likely 0% due to potential harm or lack of efficacy of mTOR inhibitors when one of the known MAPK markers, such as KRAS, are present in the molecular profile [PMID:30691487, PMID:25500057, PMID:31395751].
G. Patient N7. In a trial evaluating a novel small molecule CDK4/6 inhibitor, a patient presents with a relevant marker status of loss of function mutation in CDKN2A and CDKN2B tumor suppressor genes (upstream of CDK4/6 oncogenic cancer target) and with an amplification in CCNE1. The score for the trial drug would be 0%, because CDK4/6 inhibitors are excluded as therapy options due to drug resistance in cancer with several specific biomarkers, one of which is CCNE1 amplification [PMID: 35330128, PMID: 35304604, PMID: 30537512, PMID: 29236940, PMID: 30206110, PMID: 32404308, PMID: 30807234].
H. Patient N8. In a trial evaluating a novel small-molecule EGFR kinase inhibitor of a similar type to gefitinib, a patient presents with an EGFR T790M biomarker in their molecular profile. In such situations, the score would be 0% due to the likely resistance to these types of cancer therapies targeting EGFR conferred by this specific marker
I. Patient N9 and N10. A Clinical Trial investing only one drug (A) targeting only a single target PTCH1 pathway. Patient N9 has PTCH1 marker and another mutation that is not considered targetable by any known drugs. The score would be near 50%. Patient N10 has the same PTCH1 marker, but also presents with 3 other pathogenic markers in known cancer genes EGFR, KRAS, PTEN, and another marker that is not targetable. The best possible score for combination therapies may be roughly 80%. However, for the PTCH1 trial drug alone it would be <20%.

It should further be appreciated that the methods above are provided by way of example. Absent an explicit indication to the contrary, the disclosed steps may be modified, supplemented, omitted, and/or re-ordered without departing from the scope of this disclosure.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

1. A method for assessing eligibility of a patient to a clinical trial of a therapy comprising one or more drugs, the method comprising:
(a) providing at least one drug target for the one or more drugs, and providing a molecular profile for the patient, wherein the molecular profile is generated from analysis of a biological sample obtained from the patient and contains one or more clinically relevant biomarkers;
(b) generating a matching score for the patient based on the molecular profile and the patient's predicted response to the therapy, and setting a threshold score, wherein the generating step comprises determining impact of the therapy on the molecular profile of the patient based on the number and types of biomarkers and interactions between biomarkers and the one or more drugs of the therapy;
(c) providing a recommendation for clinical trial eligibility of the patient when the matching score for the patient passes requirements of the threshold score.

2. The method of claim 1, wherein eligibilities of a plurality of patients are assessed, and the matching score is generated for each patient of the plurality of patients.

3. The method of claim 2, wherein the threshold score is set after the matching score for each patient of the plurality of patients is generated.

4. The method of claim 3, wherein the threshold score is set at a specific percentile of a range of matching scores generated for each patient of the plurality of patients.

5. The method of claim 2, wherein the threshold score is set before the matching score for each patient of the plurality of patients is generated.

6. The method of claim 1, wherein the threshold score is set based on previously analyzed clinical trial data sets.

7. The method of claim 1, wherein the threshold score is set based on a newly obtained patient data from the clinical trial.

8. The method of claim 1, wherein the providing at least one drug target for the drug comprises providing an IC50 value of the drug for the at least one drug target.

9. The method of claim 1, wherein the patient is a cancer patient.

10. The method of claim 1, wherein the biological sample comprises a tissue, blood, plasma or serum sample.

11. The method of claim 1, wherein the analysis of the biological sample comprises performing a next-generation sequencing of nucleic acid molecules extracted from the biological sample.

12. The method of claim 11, wherein the analysis of the biological sample further comprises extracting a cell-free DNA from the biological sample.

13. The method of claim 1, wherein the clinical trial is a prospective clinical trial.

14. The method of claim 1, wherein generating the matching score further comprising determining impact of the drug on each of the one or more clinically relevant biomarkers.

15. The method of claim 1, wherein the one or more clinically relevant biomarkers is/are targeted by the one or more drugs of the therapy.

16. A system for assessing eligibility of a patient to a clinical trial of a drug, the system comprising:
a server;
a computing device in communication with the server over a network, the computing device including a processor and a memory, the memory bearing computer executable code configured to perform the steps of:
(a) providing at least one drug target for the one or more drugs, and providing a molecular profile for the patient, wherein the molecular profile is generated from analysis of a biological sample obtained from the patient and contains one or more clinically relevant biomarkers;
(b) generating a matching score for the patient based on the molecular profile and the patient's predicted response to the therapy, and setting a threshold score, wherein the generating step comprises determining impact of the therapy on the molecular profile of the patient based on the number and types of biomarkers and interactions between biomarkers and the one or more drugs of the therapy;
(c) providing a recommendation for clinical trial eligibility of the patient when the matching score for the patient passes requirements of the threshold score.

17. The system of claim 16, wherein eligibilities of a plurality of patients are assessed, and the matching score is generated for each patient of the plurality of patients.

18. The system of claim 17, wherein the threshold score is set after the matching score for each patient of the plurality of patients is generated.

19. The system of claim 17, wherein the threshold score is set at a specific percentile of a range of matching scores generated for each patient of the plurality of patients.

20. The system of claim 16, wherein the one or more clinically relevant biomarkers is/are targeted by the one or more drugs of the therapy.

21. The system of claim 16, wherein the patient is a cancer patient.

22. The system of claim 16, wherein the biological sample comprises a tissue, blood, plasma or serum sample.

23. The system of claim 16, wherein generating the matching score further comprising determining impact of the drug on each of the one or more clinically relevant biomarkers.
